(19) United States
(12) Patent Application Publication
(10) Pub. No.: US 2012/0003307 A1
(43) Pub. Date: Jan. 5, 2012

(54) LEVETIRACETAM CONTROLLED RELEASE COMPOSITION

(75) Inventors: Namdev M. Kashid, Sholapur (IN); Sunny Chopra, Chandigarh (IN); Sumit Madan, New Delhi (IN); Vinod Arora, New Delhi (IN)

(73) Assignee: RANBAXY LABORATORIES LIMITED, New Delhi, Delhi (IN)

(21) Appl. No.: 12/744,830

(22) PCT Filed: Nov. 26, 2008
(86) PCT No.: PCT/IB2008/054974

§ 371 Date: Jul. 16, 2010

(30) Foreign Application Priority Data
Nov. 29, 2007 (IN) 2495/DEL/2007

(51) Int. Cl. A61K 9/48; A61K 9/28; A61P 25/08; A61K 31/4015; A61K 9/14; A61K 9/00; A61K 9/16

(52) U.S. Cl. 424/463; 424/400; 424/474; 424/490; 427/2.21; 514/424

(57) ABSTRACT

The present invention is concerned with controlled release compositions for oral administration comprising levetiracetam; and with processes of preparing such controlled release compositions.

### FIELD OF THE INVENTION

The present invention relates to controlled release pharmaceutical compositions of levetiracetam and process for preparation thereof.

### BACKGROUND OF THE INVENTION

Levetiracetam is an antiepileptic drug which is chemically unrelated to existing antiepileptic drugs (AEDs). It is a single enantiomer, the S-form of the pyrrolidone etiracetam, chemically known as (−)-(S)-α-ethyl-2-oxo-1-pyrrolidineacetamide. The U.S. Pat. No. 4,943,639 discloses levetiracetam and also describes process for its preparation. Levetiracetam is indicated as monotherapy in the treatment of partial onset seizures with or without secondary generalization in patients from 16 years of age with newly diagnosed epilepsy and also as adjunctive therapy in the treatment of (a) partial onset seizures with or without secondary generalization in adults and children from 4 years of age with epilepsy; (b) myoclinic seizures in adults and adolescents from 12 years of age with Juvenile Myoclinic Epilepsy; and (c) primary generalized tonic-clonic seizures in adults and adolescents from 12 years of age with Idiopathic Generalized Epilepsy. Commercially it is available in strengths of 250 mg, 500 mg, 750 mg and 1000 mg film-coated immediate release tablets; 100 mg/mL oral solution and 100 mg/mL concentrate for solution for infusion under the brand name KEPPRA® by UCB S.A., Brussels, Belgium. Recently, an extended release formulation is made available in the USA from UCB Inc., under the trade name KEPPRA XR™, which contains 500 mg levetiracetam, colloidal anhydrous silica, hypromellose, magnesium stearate, polyethylene glycol 6000, polyvinyl alcohol-partially hydrolyzed, titanium dioxide, macrogol/PEG3350, and talc.

The prior art records several documents describing levetiracetam formulations. The U.S. Pat. No. 4,837,223 describes a pharmaceutical composition comprising a therapeutically effective amount of levetiracetam and a pharmaceutically acceptable solid or liquid diluent or carrier therefor. The PCT application WO 2007/012439 also relates to levetiracetam compositions with improved stability as compared to the conventional immediate release levetiracetam compositions, wherein the later may present modified kinetics in release of the active substance as time goes along, which consequently leads to slower release of the active drug substance and an earlier expiry date of the product due to reduced stability.

It is also well-documented in the art that formulating levetiracetam as controlled release dosage forms would provide various advantages over the immediate release formulations recommended for multiple dosing. The advantages being reduced fluctuations of plasma drug levels, reduced adverse effects and hence more patient compliance. Consequently, several attempts have been made in the art to formulate controlled release compositions of levetiracetam to achieve the above discussed objectives. PCT application WO 01/51033 describes a solid controlled release pharmaceutical composition for oral administration comprising the active ingredient, at least one excipient matrix, chosen among the inert matrices, the absorbent matrices, the lipidic matrices, the mixture of inert matrices and lipidic matrices, the mixture of absorbent matrices and lipidic matrices, the mixture of matrices absorbent and inert matrices; at least a entérosoluble polymer; and at least an alkalizing agent soluble in an aqueous phase under physiological pH conditions. PCT application WO 2006/088864 discloses a controlled release formulation of levetiracetam, which comprises an immediate release component and a modified release component or formulation, such that the immediate release component comprises a first population of levetiracetam and the modified release component preferably comprises a second population of levetiracetam and a controlled release constituent. It also discloses that the modified release formulation is preferably in the form of an erodable formulation, a diffusion controlled formulation or an osmotic controlled formulation and the combination of the immediate release component and the modified release component or formulation in operation deliver the active ingredient in a pulsed or bimodal manner. The PCT application WO 2006/080029 discloses an extended release tablet with the core containing levetiracetam and water dispersible rate controlling polymer and further teaches that the tablet core is optionally coated with a combination of water non-dispersible and/or water dispersible polymer. It also discloses that such a tablet provides a peak blood plasma level of levetiracetam in from about eight to sixteen hours. Another PCT application WO 2006/123357 exemplifies once-daily oral matrix based extended release pharmaceutical compositions of levetiracetam, comprising a rate-controlling agent which is optionally coated using a coating selected from (i) an active ingredient permeable coating surrounding the unit dosage form, and (ii) an active ingredient impermeable coating covering one or more surfaces but not all the surfaces of the unit dosage form.

The above discussed prior art references teaches various matrix-based extended release formulations. In the present invention, the inventors have developed easy-to-make options other than those based on matrix system, for providing a controlled release formulation comprising levetiracetam.

### SUMMARY OF THE INVENTION

In one general aspect, it relates to a controlled release pharmaceutical composition for oral administration which comprises:

(a) an immediate release core comprising of levetiracetam; and

(b) a release rate-controlling membrane coating of hydrophobic polymer(s).

In another general aspect, it relates to a controlled release pharmaceutical composition for oral administration which comprises:

(a) an immediate release core comprising of levetiracetam; and

(b) a release rate-controlling membrane coating of hydrophobic polymer(s), wherein, the said release rate-controlling membrane coating further comprises one or more of hydrophilic polymer(s), pore-forming agent(s) and plasticizer(s).

In another general aspect, it relates to a controlled release pharmaceutical composition for oral administration which comprises:

(a) an immediate release core comprising of levetiracetam; and

(b) a release rate-controlling membrane coating of hydrophobic polymer(s); wherein the said pharmaceutical composition, further comprises of a top-coat comprising of levetiracetam in immediate release form.

In another general aspect, it relates to a controlled release pharmaceutical composition for oral administration which comprises:

(a) an immediate release core comprising of levetiracetam; and

(b) a release rate-controlling membrane coating of hydrophobic polymer(s); wherein the said core is in the form of tablets, minitablets, capsules, beads, pills, pellets or granules.

In another general aspect, it relates to a controlled release pharmaceutical composition for oral administration which comprises:

(a) an immediate release core comprising of levetiracetam; and

(b) a release rate-controlling membrane coating of hydrophobic polymer(s); wherein the said pharmaceutical composition is intended to be administered once daily.

In another general aspect, it relates to a controlled release pharmaceutical composition for oral administration which comprises:

(a) an immediate release core comprising of levetiracetam; and

(b) a release rate-controlling membrane coating of hydrophobic polymer(s); wherein one or more of the said pharmaceutical composition is dispensed as a unit dosage form.

In another general aspect, it relates to a controlled release pharmaceutical composition for oral administration prepared by a process which comprises:
(a) preparing an immediate release core comprising of levetiracetam and pharmaceutically acceptable excipients selected from a group consisting of diluent(s), binder(s), lubricant(s), glidant(s) and combinations thereof; and
(b) coating the immediate release core of step (a) with a release rate-controlling membrane coating composition comprising of hydrophobic polymer(s) and one or more of hydrophilic polymer(s), pore-forming agent(s) and plasticizer(s).

In another general aspect, it relates to a controlled release pharmaceutical composition for oral administration prepared by a process which comprises:
(a) preparing an immediate release core comprising of levetiracetam and pharmaceutically acceptable excipients selected from a group consisting of diluent(s), binder(s), lubricant(s), glidant(s) and combinations thereof;
(b) coating the immediate release core of step (a) with a release rate-controlling membrane coating composition comprising of hydrophobic polymer(s) and one or more of hydrophilic polymer(s), pore-forming agent(s) and plasticizer(s); and
(c) optionally, top-coating the core of step (b) with levetiracetam in immediate release form.

In another general aspect, it relates to a controlled release pharmaceutical composition for oral administration prepared by a process which comprises:
(a) preparing an immediate release core comprising of levetiracetam and pharmaceutically acceptable excipients selected from a group consisting of diluent(s), binder(s), lubricant(s), glidant(s) and combinations thereof;
(b) coating the immediate release core of step (a) with a release rate-controlling membrane coating composition comprising of hydrophobic polymer(s) and one or more of hydrophilic polymer(s), pore-forming agent(s) and plasticizer(s); and
(c) optionally, dispensing one or more of the cores of step (b) as a unit dosage form.

In another general aspect, it relates to a controlled release pharmaceutical composition for oral administration which comprises:

(a) an immediate release core comprising of levetiracetam; and

(b) a release rate-controlling membrane coating of hydrophobic polymer(s); such that the said composition exhibits the following in-vitro dissolution profile when measured in a USP type II apparatus with 10 mesh sinkers, at 50 rpm, at a temperature of 37° C.±0.5° C. in 900 mL aqueous medium:
at most about 35% drug released in 2 hours;
at most about 55% drug released in 4 hours;
at most about 80% drug released in 8 hours; and
at least about 80% drug released in 16 hours.

### DETAILED DESCRIPTION OF THE INVENTION

The term “controlled release pharmaceutical composition”, as referred to herein, is defined to mean oral pharmaceutical compositions which when administered releases the active medicament at a relatively constant rate and provide plasma concentrations of the active medicament that remain substantially invariant with time within the therapeutic range of the active medicament over a 24-hour period and encompasses “prolonged release”, “extended release”, “modified release”, “delayed release” and “sustained release” compositions. The compositions according to the present invention deliver a therapeutically effective amount of levetiracetam to a patient for 24 hours following a once-daily administration. The term “therapeutically effective amount” intends to describe an amount of the active agent which stops or reduces the progress of the condition to be treated or which otherwise completely or partly cures or acts palliatively on the condition. Levetiracetam or a pharmaceutically acceptable salt or derivative thereof may be present in an amount about 100 mg to about 1600 mg. The recommended dose of KEPPRA XR™ may be considered as a standard dose.

The controlled release pharmaceutical composition, as described herein, include multiparticulate systems, ion-exchange resin beads, pulsatile devices, multilayered tablets, osmotic systems, reservoir based systems and membrane controlled systems. The compositions are administered as unit dosage forms and may be in the form of granules, tablets, pellets or capsules.

The “immediate release core”, as used herein, comprises levetiracetam and one or more of pharmaceutically acceptable excipients. The “immediate release core” may be in the form of tablets, minitablets, capsules, granules, pellets, beads or pills.

The term “pharmaceutically acceptable excipients”, as recited herein, includes conventional pharmaceutical additives known in the art, such as diluent(s), binder(s), lubricants(s), granulating solvent(s), glidants(s) or combinations thereof.

Diluents that may be used may be exemplified, but are not limited to, saccharides like lactose, dextrose, sucrose, fructose, maltose; sugars like mannitol, erythritol, sorbitol, xylitol and lactitol; cellulose derivatives like powdered cellulose, microcrystalline cellulose; dicalcium phosphate, tribasic calcium phosphate, calcium sulphate, calcium carbonate, kaolin and the like. The diluent may comprise from about 1% to about 50%, preferably from about 1% to about 20% by weight of the controlled release pharmaceutical composition. In one embodiment, the diluent is microcrystalline cellulose.

Binders that may be used include, but are not limited to, starch derivatives like corn starch and pregelatinized starch; cellulose ethers such as carboxymethyl cellulose, methylcellulose, hydroxypropyl cellulose, hydroxypropyl methylcellulose; carboxy vinyl polymers like carbomers; acrylates such as Eudragits; polyvinylpyrrolidone, polyvinylpyrrolidone/vinyl acetate copolymer; xanthan gum, guar gum and other such materials routinely used in the art of solid dosage form manufacturing. The binder may be present in an amount varying from about 0.1% to about 10% by weight of the controlled release pharmaceutical composition. In one embodiment, the binder is polyvinylpyrrolidone.

Lubricants include magnesium stearate, calcium stearate, zinc stearate, sodium stearyl fumarate, powdered stearic acid, magnesium oleate, calcium palmitate, potassium laureate, sodium suberate, vegetable oil, mineral oil, and the like. Glidants may be selected from talc, colloidal silicon dioxide, corn starch and the like. The lubricant and glidants may be used in an amount of 0.1% to 2% by weight of the controlled release pharmaceutical composition. In one embodiment, the lubricant and glidants used are magnesium stearate and talc.

Suitable granulating solvents may be used which include without limitation, water, ethanol, methanol, isopropyl alcohol, methylene chloride, acetone and the like.

The controlled release pharmaceutical composition of the invention includes membrane-moderated or reservoir systems, wherein a reservoir of the active ingredient is surrounded by a release rate-controlling membrane coating. Without being bound by theory, it may be stated that the active ingredient traverses the membrane by mass transport mechanisms well known in the art, including but not limited to dissolution in the membrane followed by diffusion across the membrane or diffusion through liquid-filled pores within the membrane. These individual reservoir system dosage forms may be large, as in the case of a tablet or capsules containing a single large reservoir, or multiparticulates, as in the case of a capsule containing a plurality of reservoir particles, each individually coated with a membrane. The coating may be non-porous, yet permeable to the active ingredient (for example levetiracetam may diffuse directly through the membrane), or it may be porous.

The “release rate-controlling membrane coating”, as described herein, comprises of hydrophobic polymer(s). The amount of polymer(s) relative to levetiracetam depends upon the rate of drug release required and also upon the type and molecular weight of the polymer and other excipients present in the formulation. The hydrophobic polymers which may be used include polymers such as ethyl cellulose, cellulose acetate, acrylic acid polymers and copolymers, high molecular weight polyvinyl alcohols and waxes such as fatty acids and glycerides. The amount of hydrophobic polymer(s) may be present in an amount varying from about 1% to about 20% by weight of the controlled release pharmaceutical composition or from about 30% to about 90%; preferably about 40% to about 70% of the of the release rate-controlling membrane coating. In one embodiment, the hydrophobic polymer used is ethylcellulose. Ethylcellulose used, may be the one available from Dow Chemical under the trade name Ethocel®.

The “release rate-controlling membrane coating”, as described herein, may further comprise of hydrophilic polymer(s). Hydrophilic polymer(s) suitable for use in the controlled release pharmaceutical composition may include without limitation, cellulosic polymers such as hydroxypropyl methylcellulose, methylcellulose, hydroxymethyl cellulose carboxymethyl cellulose, sodium carboxymethyl cellulose, hydroxypropylcellulose and hydroxyethylcellulose. Other hydrophilic polymers include natural or partially or totally synthetic hydrophilic gums such as acacia, gum tragacanth, locust bean gum, guar gum, karaya gum; proteinaceous substances such as agar, pectin, carrageenan, galactomannan and alginates; polyvinylpyrrolidone; vinyl acetate copolymers; starch and starch based polymers; polysaccharides; methacrylic acid copolymers; maleic anhydride/methyl vinyl ether copolymers; carboxypolymethylene; gelatin; casein, zein; bentonite; magnesium aluminium silicate; polyethylene oxide and other hydrophilic polymers known to those skilled in the art or a derivative or a mixture thereof. The hydrophilic polymer(s) may comprise about 20% to about 40% by weight of the release rate-controlling membrane coating.

In one embodiment, the release rate-controlling membrane coating composition comprises of a combination of ethylcellulose and hydroxypropyl methylcellulose.

The release rate-controlling membrane coating may further comprise of a pore-forming agent. Pore-forming agents that may be used include without limitation, water-soluble compounds and hydrophilic polymers. The pore-forming agents that may be used in the present invention may be selected from the group consisting of alkali metal salts, e.g., sodium chloride, sodium bromide and the like; alkaline earth metals, e.g., calcium phosphate, calcium nitrate and the like; transition metal salts, e.g., ferric chloride, ferrous sulfate and the like; carbohydrates, for e.g., glyceraldehydes, erythrose, ribose, arabinose, xylose, glucose, mannose, galactose, maltose, lactose, sucrose and the like; and sugar alcohols, e.g., mannitol and the like.

The release rate-controlling membrane coating may further comprise of other conventionally used coating additives, selected from the group comprising of plasticizers, coloring agents, lubricants/glidants, and the like. Plasticizers that may be used include without limitation dibutyl sebacate, triethyl citrate, acetylated triacetin, tributyl citrate, glycerol tributyrate, monoglyceride, rape oil, olive oil, sesame oil, acetyltributylcitrate, acetyltriethylcitrate, glycerin sorbitol, diethyl oxalate, diethyl phthalate, diethyl malate, diethyl fumarate, dibutyl succinate, diethyl malonate, dioctyl phthalate, and the like. In one embodiment, dibutyl sebacate and triethyl citrate are used as plasticizers.

The release rate-controlling membrane coating composition may further contain one or more solvents. Suitable solvents for the release rate-controlling membrane coating composition may comprise of various solvents including, but not limited to, isopropyl alcohol (IPA), water, methylene chloride and mixtures thereof. Various combinations of solvents may be used such as isopropyl alcohol and water, isopropyl alcohol and methylene chloride, and the like.

The controlled release pharmaceutical composition for oral administration may be prepared by a process, comprising the following steps:
(a) preparing an immediate release core comprising of levetiracetam; and
(b) coating the said immediate release core with a release rate-controlling membrane coating composition comprising of hydrophobic polymer(s).

The immediate release core comprising levetiracetam as described herein may be formulated following any conventional techniques known in the art, namely dry granulation, aqueous or non-aqueous wet granulation, direct compression and pelletization.

The immediate release core may further be coated by a non-functional seal coat, comprising of conventionally known film-coating excipients. An optional seal coat may be applied between the immediate release core and the release rate-controlling membrane coating. The seal coating layer is applied to the immediate release cores to prevent sticking of the cores during the process of coating and to prevent migration of the drug into the release rate-controlling membrane during manufacturing. In one embodiment, a thin aqueous layer of hydroxypropyl methylcellulose and talc is used as a seal coating layer.

Alternatively, the controlled release pharmaceutical compositions as discussed herein may further comprise a top-coat of levetiracetam and a hydrophilic polymer as described hereinbefore, wherein the active ingredient is released practically immediately upon ingestion and thus ensures a rapid onset of action.

The immediate release core may be coated with the release rate-controlling membrane coating using various techniques, viz. conventional coating techniques such as perforated pan, fluidized bed technique or spraying. The weight of the release rate-controlling membrane coating ranges from about 3% to about 15%, preferably from about 4% to about 12% by weight of the controlled release pharmaceutical composition. The rate of release of the active ingredient from the composition is approximately inversely proportional with the thickness of the release rate controlling membrane coating.

One or more of the controlled release pharmaceutical composition as discussed herein may be dispensed as a unit dosage form. The compositions may be filled in hard-gelatin capsules such that a therapeutically effective amount of the active ingredient is available per dosage form. Alternately, one or more of the controlled release pharmaceutical composition as referred to herein may be packed into an appropriate packaging for single dose administration.

In one embodiment, a controlled release pharmaceutical composition is prepared by:
(a) preparing an immediate release core comprising of levetiracetam and pharmaceutically acceptable excipients comprising of diluent(s), binder(s), lubricant(s) and glidant(s); and
(b) coating the immediate release core of step (a) with a release rate-controlling membrane coating composition comprising of hydrophobic polymer(s), hydrophilic polymer(s), plasticizer(s) and lubricant(s).

In one embodiment, a controlled release pharmaceutical composition is prepared by:
(a) preparing an immediate release core comprising of levetiracetam and pharmaceutically acceptable excipients comprising of diluent(s), binder(s), lubricant(s) and glidant(s); and
(b) coating the immediate release core of step (a) with a release rate-controlling membrane coating composition comprising of hydrophobic polymer(s), pore-forming agent(s), plasticizer(s) and lubricant(s).

In one embodiment, a controlled release pharmaceutical composition is prepared by:
(a) preparing an immediate release core comprising of levetiracetam and pharmaceutically acceptable excipients comprising of diluent(s), binder(s), lubricant(s) and glidant(s);
(b) coating the immediate release core of step (a) with a release rate-controlling membrane coating composition comprising of hydrophobic polymer(s), hydrophilic polymer(s), plasticizer(s) and lubricant(s); and
(c) filling one or more of the coated cores of step (b) into a hard gelatin capsule of suitable size using appropriate tooling.

In one embodiment, a controlled release pharmaceutical composition is prepared by:
(a) preparing an immediate release core comprising of levetiracetam and pharmaceutically acceptable excipients comprising of diluent(s), binder(s), lubricant(s) and glidant(s);
(b) coating the immediate release core of step (a) with a release rate-controlling membrane coating composition comprising of hydrophobic polymer(s), pore-forming agent(s), plasticizer(s) and lubricant(s); and
(c) filling one or more of the coated cores of step (b) into a hard gelatin capsule of suitable size using appropriate tooling.

In one embodiment, a controlled release pharmaceutical composition is prepared by:
(a) preparing an immediate release core comprising of levetiracetam and pharmaceutically acceptable excipients comprising of diluent(s), binder(s), lubricant(s) and glidant(s);
(b) coating the immediate release core of step (a) with a release rate-controlling membrane coating composition comprising of hydrophobic polymer(s), pore-forming agent(s), plasticizer(s) and lubricant(s); and
(c) top-coating the coated cores of step (b) with levetiracetam dispersed in a polymeric solution.

In one embodiment, a controlled release pharmaceutical composition is prepared by:
(a) preparing an immediate release core comprising of levetiracetam and pharmaceutically acceptable excipients comprising of diluent(s), binder(s), lubricant(s) and glidant(s);
(b) coating the immediate release core of step (a) with a release rate-controlling membrane coating composition comprising of hydrophobic polymer(s), hydrophilic polymer(s), plasticizer(s) and lubricant(s); and
(c) top-coating the coated cores of step (b) with levetiracetam dispersed in a polymeric solution.

In one embodiment, a controlled release pharmaceutical composition is prepared by a process, comprising the following steps:
(a) levetiracetam is blended with a diluent and granulated using an aqueous solution of a binder in a suitable mixer granulator;
(b) the granules of step (a) are dried and sized;
(c) the sized granules of step (b) are blended with one or more of binders, lubricants and/or glidants;
(d) the blend of step (c) is filled into a capsule, or compressed into a tablet using appropriate tooling;
(e) the capsule or tablet of step (d) is optionally coated using a non-functional seal coating composition;
(f) the capsule or tablet of step (d) or (e) is coated using a release rate-controlling membrane coating composition comprising of hydrophobic polymer(s), hydrophilic polymer(s), plasticizer(s) and lubricant(s);
(g) the coated capsules or tablets of step (f) is cured under appropriate conditions; and
(h) the cured capsule or tablet of step (g) is optionally top coated with levetiracetam dispersed in a polymeric solution.

In one embodiment, a controlled release pharmaceutical composition is prepared by a process, comprising the following steps:
(a) levetiracetam is blended with a diluent and granulated using an aqueous solution of a binder in a suitable mixer granulator;
(b) the granules of step (a) are dried and sized;
(c) the sized granules of step (b) are blended with one or more of binders, lubricants and/or glidants;
(d) the blend of step (c) is filled into a capsule, or compressed into a tablet using appropriate tooling;
(e) the capsule or tablet of step (d) is optionally coated using a non-functional seal coating composition;
(f) the capsule or tablet of step (d) or (e) is coated using a release rate-controlling membrane coating composition comprising of hydrophobic polymer(s), pore-forming agent(s), plasticizer(s) and lubricant(s);
(g) the coated capsules or tablets of step (f) is cured under appropriate conditions; and
(h) the cured capsule or tablet of step (g) is optionally top coated with levetiracetam dispersed in a polymeric solution.

From the above it is apparent that various modifications and combinations of the formulations detailed in the text may be made without departing from the spirit and scope of the invention. The invention as described herein may be illustrated by the following examples but is not to be construed to be limiting by them.

### EXAMPLES 1-6

| | | Quantity (mg) | | | | | |
|---|---|---|---|---|---|---|---|
| SN | Ingredients | Example 1 | Example 2 | Example 3 | Example 4 | Example 5 | Example 6 |

Intragranular

| | | | | | | | |
|---|---|---|---|---|---|---|---|
| 1. | Levetiracetam | 1000.00 | 125.00 | 750.00 | 500.00 | 1000.00 | 500.00 |
| 2. | Polyvinyl | 70.00 | 8.50 | 10.00 | 10.00 | 75.00 | 37.50 |
| | pyrrolidone | | | | | | |
| 3. | Microcrystalline | 120.00 | 20.00 | 50.00 | 50.00 | 120.00 | 60.00 |
| | cellulose | | | | | | |
| 4. | Water | q.s. | q.s. | q.s. | q.s. | q.s. | q.s. |

Extragranular

| | | | | | | | |
|---|---|---|---|---|---|---|---|
| 5. | Microcrystalline | 50.00 | 10.00 | — | — | 65.00 | 32.50 |
| | cellulose | | | | | | |
| 6. | Talc | 7.50 | 0.75 | 2.00 | 2.00 | 7.50 | 3.75 |
| 7. | Magnesium stearate | 7.50 | 0.75 | 2.00 | 2.00 | 7.50 | 3.75 |

Seal coating

| | | | | | | | |
|---|---|---|---|---|---|---|---|
| 8. | Hydroxypropyl | — | — | 41.00 | 40.00 | — | — |
| | methylcellulose | | | | | | |
| 9. | Talc | — | — | 4.00 | 4.00 | — | — |
| 10. | Water | — | — | q.s. | q.s. | — | — |

Release rate-controlling membrane coating

| | | | | | | | |
|---|---|---|---|---|---|---|---|
| 11. | Ethyl cellulose | 90.00 | 10.50 | 57.00 | 42.00 | 71.40 | 35.70 |
| 12. | Dibutyl sebacate | 6.50 | 0.80 | 4.00 | 3.00 | 15.30 | 7.65 |
| 13. | Triethyl citrate | 19.00 | 2.50 | 10.00 | 10.00 | 10.20 | 5.10 |
| 14. | Hydroxypropyl | 38.00 | 6.00 | 24.00 | — | 30.60 | 15.30 |
| | methylcellulose | | | | | | |
| 15. | Lactose | — | — | — | 18.00 | — | — |
| | monohydrate | | | | | | |
| 16. | Water | q.s. | q.s. | q.s. | q.s. | q.s. | q.s. |

Top coating

| | | | | | | | |
|---|---|---|---|---|---|---|---|
| 17. | Levetiracetam | — | — | 250.00 | — | — | — |
| 18. | Hydroxypropyl | — | — | 25.00 | — | — | — |
| | methylcellulose | | | | | | |
| 19. | Talc | — | — | 2.5 | — | — | — |
| 20. | Water | — | — | q.s. | — | — | — |

| | | | | | | |
|---|---|---|---|---|---|---|
| Total weight | 1438.50 | 184.80 | 1231.50 | 681.00 | 1402.5 | 701.25 |

### Procedure

### Example 1

Levetiracetam was blended with intragranular microcrystalline cellulose and granulated using an aqueous solution of polyvinylpyrrolidone in rapid mixer granulator. The granules so obtained were dried and subjected to sizing. The sized granules thus obtained were blended with extragranular microcrystalline cellulose, magnesium stearate and talc. The blend so formed was compressed into capsule-shaped tablets. The compressed tablets were loaded in a coating pan and coated using an aqueous dispersion of ethylcellulose, hydroxypropyl methylcellulose, triethyl citrate and dibutyl sebacate until desired weight gain was achieved. The coated tablets were cured for 10-16 hours at 40° C.

### Example 2

Levetiracetam was blended with intragranular microcrystalline cellulose and granulated using an aqueous solution of polyvinylpyrrolidone in rapid mixer granulator. The granules so obtained were dried and subjected to sizing. The sized granules thus obtained were blended with extragranular microcrystalline cellulose, magnesium stearate and talc. The blend so formed was compressed into tablets. The compressed tablets were loaded in a coating pan and coated using an aqueous dispersion of ethylcellulose, hydroxypropyl methylcellulose, triethyl citrate and dibutyl sebacate until desired weight gain is achieved. The coated tablets were cured for 10-16 hours at 40° C. Four of the finished tablets so obtained were filled into size “00” hard gelatin capsules using appropriate tooling. Two such capsules were packed into appropriate packaging suitable for single dose administration.

### Example 3

Levetiracetam was blended with microcrystalline cellulose and granulated using an aqueous solution of polyvinylpyrrolidone in rapid mixer granulator. The granules so obtained were dried and subjected to sizing. The sized granules thus obtained were blended with magnesium stearate and talc. The blend so formed was filled into size “00” hard gelatin capsules using appropriate tooling. The capsules so obtained were seal-coated using an aqueous coating composition comprising of hydroxypropyl methylcellulose and talc. The seal coated capsules were loaded in a coating pan and coated using an aqueous dispersion of ethylcellulose, hydroxypropyl methylcellulose, triethyl citrate and dibutyl sebacate until desired weight gain was achieved. The coated capsules were cured for 10-16 hours at 40° C. The cured capsules so obtained were further top-coated with a coating composition comprising of levetiracetam, hydroxypropyl methylcellulose and talc.

### Example 4

Levetiracetam was blended with microcrystalline cellulose and granulated using an aqueous solution of polyvinylpyrrolidone in rapid mixer granulator. The granules so obtained were dried and subjected to sizing. The sized granules thus obtained were blended with magnesium stearate and talc. The blend so formed was filled into size “00” hard gelatin capsules using appropriate tooling. The capsules so obtained were seal-coated using an aqueous coating composition comprising of hydroxypropyl methylcellulose and talc. The seal coated capsules were loaded in a coating pan and coated using an aqueous dispersion of ethylcellulose, lactose monohydrate, triethyl citrate and dibutyl sebacate until desired weight gain was achieved. The coated capsules were cured for 10-16 hours at 40° C. Two such capsules were packed into appropriate packaging suitable for single dose administration.

### Example 5

Levetiracetam was blended with intragranular microcrystalline cellulose and granulated using an aqueous solution of polyvinylpyrrolidone in rapid mixer granulator. The granules so obtained were dried and subjected to sizing. The sized granules thus obtained were blended with extragranular microcrystalline cellulose, magnesium stearate and talc. The blend so formed was compressed into tablets. The compressed tablets were loaded in a coating pan and coated using an aqueous dispersion of ethylcellulose, hydroxypropyl methylcellulose, triethyl citrate and dibutyl sebacate until desired weight gain was achieved. The coated tablets were cured for 16 hours at 40° C.

### Example 6

These tablets can be prepared by blending levetiracetam with intragranular microcrystalline cellulose and granulated using an aqueous solution of polyvinylpyrrolidone in rapid mixer granulator. The granules can then be dried and subject to sizing. The sized granules thus obtained are blended with extragranular microcrystalline cellulose, magnesium stearate and talc. The blend so formed can be compressed into tablets. The compressed tablets can be loaded in a coating pan and coated using an aqueous dispersion of ethylcellulose, hydroxypropyl methylcellulose, triethyl citrate and dibutyl sebacate until desired weight gain is achieved. The coated tablets can be cured for 10-16 hours at 40° C.

Tablets of Example 5 were subjected to in-vitro dissolution studies in a USP type II apparatus with 10 mesh sinkers, at 50 rpm, at a temperature of 37° C.±0.5° C. in 900 mL aqueous medium. Aliquot of sample was withdrawn at predetermined time intervals and replaced with an equal amount of fresh media. Samples were processed and analysed suitably. Dissolution profiles of these tablets are provided in Table 1.

TABLE 1
In-vitro release patter nof levetiracetam from controlled release tablets
prepared as per composition of Example 5 in USP II apparatus in
900 mL of aqueous solution at 50 rpm at a temperature of
37° C. ± 0.5° C., using 10 mesh sinkers.

| Time | Percent of levetiracetam released from the |
|---|---|
| (h) | composition prepared as per Example 5 |
| 1 | 15 ± 1.6 |
| 2 | 26 ± 1.2 |
| 4 | 41 ± 1.8 |
| 6 | 53 ± 2.0 |
| 8 | 62 ± 2.2 |
| 10 | 70 ± 2.5 |
| 12 | 76 ± 2.5 |
| 16 | 84 ± 2.5 |
| 20 | 90 ± 2.4 |
| 24 | 93 ± 2.0 |

We claim:

1. A controlled release pharmaceutical composition which comprises:
(a) an immediate release core comprising of levetiracetam; and
(b) a release rate-controlling membrane coating of hydrophobic polymer(s).

2. The controlled release pharmaceutical composition according to claim 1, wherein the release rate-controlling membrane coating further comprises one or more of hydrophilic polymer(s), pore-forming agent(s) and plasticizer(s)

3. The controlled release pharmaceutical composition according to claim 1, wherein the hydrophobic polymer(s) is selected from the group consisting of ethyl cellulose, cellulose acetate, acrylic acid polymers and copolymers, high molecular weight polyvinyl alcohols and waxes such as fatty acids and glycerides, or combinations thereof.

4. The controlled release pharmaceutical composition according to claim 3, wherein the hydrophobic polymer(s) is ethylcellulose.

5. The controlled release pharmaceutical composition according to claim 1, wherein the release rate-controlling membrane coating ranges from about 3% to about 15% by weight of the controlled release pharmaceutical composition.

6. The controlled release pharmaceutical composition according to claim 3, wherein the hydrophobic polymer(s) is present in an amount from about 30% to about 90% by weight of the release rate-controlling membrane coating.

7. The controlled release pharmaceutical composition according to claim 2, wherein the hydrophilic polymer(s) is selected from the group consisting of hydroxypropyl methylcellulose, methylcellulose, hydroxymethyl cellulose carboxymethyl cellulose, sodium carboxymethyl cellulose, hydroxypropylcellulose hydroxyethylcellulose, natural or partially or totally synthetic hydrophilic gums such as acacia, gum tragacanth, locust bean gum, guar gum, karaya gum; proteinaceous substances such as agar, pectin, carrageenan, galactomannan and alginates; polyvinylpyrrolidone; vinyl acetate copolymers; starch and starch based polymers; polysaccharides; methacrylic acid copolymers; maleic anhydride/methyl vinyl ether copolymers; carboxypolymethylene; gelatin; casein, zein; bentonite; magnesium aluminium silicate; and polyethylene oxide.

8. The controlled release pharmaceutical composition according to claim 2, wherein the hydrophilic polymer(s) comprise about 20% to about 40% by weight of the release rate-controlling membrane coating.

9. The controlled release pharmaceutical composition according to claim 2, wherein the pore-forming agent(s) is selected from alkali metal salts; alkaline earth metals;
transition metal salts; carbohydrates; and sugar alcohols.

10. The controlled release pharmaceutical composition according to claim 1, wherein the said composition exhibits the following in-vitro dissolution profile when measured in a USP type II apparatus with 10 mesh sinkers, at 50 rpm, at a temperature of 37° C.±0.5° C. in 900 mL aqueous medium:
at most about 35% drug released in 2 hours;
at most about 55% drug released in 4 hours;
at most about 80% drug released in 8 hours; and
at least about 80% drug released in 16 hours.

11. The controlled release pharmaceutical composition according to claim 1, wherein the said pharmaceutical composition, further comprises of a top-coat comprising of levetiracetam in immediate release form.

12. The controlled release pharmaceutical composition according to claim 1, wherein the immediate release core is in the form of tablets, minitablets, capsules, beads, pills, pellets or granules.

13. The controlled release pharmaceutical composition according to claim 1, wherein the said pharmaceutical composition is intended to be administered once daily.

14. The controlled release pharmaceutical composition according to claim 1, wherein one or more of the said pharmaceutical composition is dispensed as a unit dosage form.

15. The controlled release composition according to claim 1, wherein a seal-coat is present between the immediate release core and the release rate-controlling membrane coating.

16. The controlled release composition according to claim 15, wherein the seal-coat comprises hydroxy propyl methylcellulose and talc.

17. A process for the preparation of the controlled release pharmaceutical composition according to claim 1, which comprises of the following steps:
(a) preparing an immediate release core comprising of levetiracetam and pharmaceutically acceptable excipients selected from a group consisting of diluent(s), binder(s), lubricant(s), glidant(s) and combinations thereof; and
(b) coating the immediate release core of step (a) with a release rate-controlling membrane coating composition comprising of hydrophobic polymer(s) and one or more of hydrophilic polymer(s), pore-forming agent(s) and plasticizer(s).

18. The process for the preparation of the controlled release pharmaceutical composition according to claim 15, which further comprises top-coating the core of step (b) with levetiracetam in immediate release form.

19. The process for the preparation of the controlled release pharmaceutical composition according to claim 17, which further comprises dispensing one or more of the cores of step (b) a; a unit dosage form.
